(19) United States
(12) Patent Application Publication
(10) Pub. No.: US 2005/0015121 A1
(43) Pub. Date: Jan. 20, 2005

(54) Light wand for healing tissue

(76) Inventor: Sherry L. Molina, Charlotte, NC (US)

Correspondence Address: MORGAN & FINNEGAN, L.L.P., 3 WORLD FINANCIAL CENTER, NEW YORK, NY, 10281-2101, US

(21) Appl. No.: 10/855,058

(22) Filed: May 27, 2004

Related U.S. Application Data
(60) Provisional application No. 60/473,965, filed on May 27, 2003.
(60) Provisional application No. 60/479,053, filed on Jun. 16, 2003.

(51) Int. Cl. A61N 5/06

(52) U.S. Cl. 607/88

(57) ABSTRACT

A light wand includes light emitting diodes that transmit monochromatic light for a therapeutic response. The monochromatic light provides healing and relieves pain from such conditions as, for example, abrasions, cuts, wounds, acne, and other skin conditions. Pulsed light and/or a constant light stream are emitted from the wand under the user's control. Intensity of the light energy is also controlled by the user through a control pad on the light wand. The light wand is portable and dimensioned for consumer use.

### CROSS REFERENCE TO RELATED APPLICATIONS

This application claims priority to U.S. Provisional application Ser. No. 60/473,965 filed on May 27, 2003, and on U.S. Provisional application Ser. No. 60/479,053 filed on Jun. 16, 2003.

### FIELD OF THE INVENTION

This invention generally relates to a medical device that delivers light for healing tissue. More particularly, the present invention is directed to a light wand that contains monochromatic light emitting diodes that deliver light energy to a tissue region for the healing and treatment of various medical conditions such as wound healing, skin conditions, and relief of acute or chronic pain.

### BACKGROUND

Traditionally, laser light is used in medical procedures involving living tissue. Lasers have been used to cauterize tissue, to remove scars, blemishes and warts, and to surgically cut tissue. The use of lasers to heal wounds involves exposure of heat to the living tissue. This exposure can negatively affect tissue surrounding the treatment site. In addition, lasers are very expensive devices and require a large amount of power, which predominantly preclude their use outside a hospital or medical setting.

Light emitting diodes (LEDs) are not widely accepted as suitable for medical use because of their low intensity and limited power output. LED's power output is measured in milli-watts and do not deliver enough power to damage tissue. LEDs do deliver enough energy to stimulate a response from the body on a cellular level. LED's do not have the same risk of accidental eye damage that lasers do.

Current state of the art use of LEDs for bio-activation of living tissue has many disadvantages. For example, in U.S. Pat. No. 6,663,659 to McDaniel, bio-activation or bio-inhibition is done by using a multi-chromatic radiation. Using multiple colored LEDs is not as effective in tissue healing as using monochromatic light in the red range. Also, the apparatus in the McDaniel patent does not allow a user to adjust frequencies or energy densities of the light energy emitted by the LEDs. This deficiency can result in over-exposing or under-exposing a treatment site.

Dr. Harry T. Whelan, a researcher in the field of LED technology at the Medical College of Wisconsin through a study funded by NASA developed a system shaped like a box. The box has a flat array of LEDs arranged in rows on top of the small box. In the study aimed to learn about the healing of mouth sores, a nurse practitioner would place the light box of red LED's on the outside of the patient's cheek for about one minute each day. The red light penetrated to the inside of the mouth, where it seems to promote healing and prevent further sores in the patient's mouth. The Whelan system has many disadvantages. This system does not having an automatic shut-off device to prevent the treatment site from being over or under exposed. In addition, the rows of LEDs require the user to move the LEDs back and forth over a treatment site in order to cover a treatment site area.

Thus, there remains a need for a device that provides the benefits of light exposure with user control over the light energy. In addition, a need exists for a device that is flexible enough to provide varying frequencies and energy densities. Finally, a need exists for a light emitting device that is easy to use, portable, and able to uniformly cover a treatment site area with the light energy.

### SUMMARY OF THE INVENTION

The present invention provides for a light wand that utilizes monochromatic light energy from light emitting diodes (LEDs). Monochromatic light in the red range having a frequency between about 600 nanometers (nm) to about 980 nm is useful in the relief of acute or chronic pain and wound healing of tissue. A control unit offers control over a range of varying frequencies and energy densities of the light energy from the LEDs. Adjustment of frequencies or energy densities of the light energy emitted by the LEDs is provided by a control unit to avoid over-exposing or under-exposing a treatment site.

The LEDs are configured in a circular array to provide uniform coverage of the emitting light energy.

The present invention is also directed to a light wand that contains monochromatic light emitting diodes that deliver light energy to a tissue region for the healing and treatment of various medical conditions such as wound healing, skin conditions, and relief of acute or chronic pain.

The present invention is also portable and easy to use. Power is supplied by an internal battery or through an AC adapter.

Light therapy from the light wand is emitted either in a pulsed or a constant light stream or beam or beams. An automatic shut off is provided in the control unit to avoid over or under exposure of the treatment site. This automatic shut off turns the device off when the desired intensity is reached. This device is controlled by a microprocessor that prevent over exposure to the treatment site. The microprocessor adjusts the timing of exposure as the internal or external power source weakens or fluctuates. For an internal battery source, as the battery weakens, the microprocessor adjusts the time of exposure so that the desired intensity of the LEDs is consistently achieved. If the power source is too weak to deliver the desired intensity, the device warns the user of this condition, and will prevent delivery of an undesirable dose. The present invention also has the ability to control time of light energy delivery and intensity of light energy delivery.

These, and other aspects of the apparatus of the present invention, are described in the following brief and detailed description of the drawings.

### BRIEF DESCRIPTION OF THE DRAWINGS

FIG. 1 is a top perspective view a light wand.

FIG. 2 is a bottom perspective view of the wand in FIG. 1.

FIG. 3A is a top view of the wand in FIG. 1 illustrating a light emitting diode pattern with red diodes and infrared diodes.

FIG. 3B is an enlarged view of the wand in FIG. 3A illustrating an alternative light emitting diodes pattern.

FIG. 4 is a top view of the wand in FIG. 3A illustrating a light emitting diode pattern with only red diodes.

FIG. 5A is a schematic of the power switch for the light wand in FIG. 1.

FIG. 5B is a schematic of a control unit for the light wand in FIG. 1.

### DETAILED DESCRIPTION

The light wand of the present invention can be any shape or size. Preferably, the wand is small enough to be carried. For example, the wand can be, but is not limited to the following dimensions: approximately 2½ inches×7 inches×1 inch. The light wand is shaped for ease of use. Light emitting diodes (LEDs) are used to emit light energy for the promotion of tissue healing and other beneficial results. The light energy from LEDs can assist with various skin conditions, for example, acne, rosacea, and eczema and reduce inflammation in swollen gums associated with baby teething.

Monochromatic light from LEDs is focused light energy that isolates on the most potent frequencies for the relief of acute or chronic pain and wound healing. Cell tissue responds best to certain frequencies that appear to be with the infrared spectrum, such as 660 nanometers (nm), 660 nm, 880 nm, 940 nm and 950 nm. The water and blood content within the body tissue restrict full absorption of light frequencies outside the range of 600-980 nm.

One frequency of light may be a primary resonant frequency for the body, while the others may be wasted harmonics. A single wavelength within the middle of the spectrum (e.g. 660 nm) is the most resonant frequency to the human tissue. An application of a 660 nm light beam for several minutes to a wound approximately the size of a half of dollar every two hours can (within a day or two) stimulate the generation of new skin without scabbing or forming scar tissue.

Photons emitted by the monochromic LEDs are absorbed by the skin and underlying tissue, triggering biological changes within the body in a process know as “photo-bio-stimulation.” Monochromatic light increases oxygen and blood flow, wound healing and stimulates nerve functioning as well as facilitating pain reduction and muscular relaxation. Light in the red spectrum and the infrared spectrum enhances and speeds up certain cellular metabolic processes, such as activity of mitochondria (cellular organelles outside the nucleus that convert stored chemical energy into a more usable form), charging the electrophysiological (electrical aspect) properties of the cell membrane and activating enzymes which turn into activate key chemical reactions.

Light energy is used as trigger for the rearrangement of cellular metabolism. There are photo acceptors at the molecular and cellular level that, when triggered, cause a series of biological actions. For example, DNA and RNA synthesis, protein and collagen synthesis and cellular proliferation are all increased when exposed to monochromatic light. The results of these biological actions are a rapid regeneration, normalization and healing of damaged cells tissue.

Exposure to monochromic LED's (light) energy is an effective therapy that works in harmony with the body's own healing and pain relieving mechanisms. There are no known harmful side effects from the LED's light. Light technology can also be used with hard-to-heal wounds, such as diabetic ulcers, serious burn and severe oral sores caused by chemotherapy and radiation.

The light energy from monochromic LEDs stimulates the production of collagen, the most common protein found in the body. This essential protein is used to repair damaged tissues and replace old tissue. Collagen is the substance that holds cells together and has a high degree of elasticity. By increasing collagen production, less scar tissue is formed at the site of injury. The LED light energy also increases circulation by increasing the actual formation of the new capillaries, which are additional blood vessels that replace damaged ones. New capillaries that carry oxygen and nutrients are needed for healing and allow waste to be carried away for healthy skin to grow.

LED light energy using monochromic light also stimulates the lymphatic system, helping to eliminate toxins and excess fluids from the issue. The diameter of the lymph vessel (a vascular duct) accompanying lymph flow can be doubled with the use of light therapy that can increase the number of white blood cells in the blood circulation.

Monochromic LED light therapy can also stimulate the release of adenosine triphosphate (ATP). This chemical is the body's major carrier of energy to the cells. Increased amounts of this chemical allows cells to accept nutrients faster, and get rid of waste products faster by increasing the energy in the cell. Increases of RNA and DNA synthesis also are available with light therapy that helps older or damaged cells to be replaced faster. Further biological effects of LED light therapy include the following. The LED light can reduce excitability of the nerves tissue. The photon light energy enters the body as negative ions that require the body to send positive ions, calcium among others, to flow to the area being treated. These ions assist in regulating the nerves, thereby relieving pain. The LED light can also stimulates the activity of the fibroblasts in the connective tissue. They are capable of forming collagen which aids in the repair process. LED light stimulates proper tissue granulation, which is part of the healing process of inflamed tissue, such as acne breakouts. Monochromic LED light stimulates biological processes. Just as plants are exposed to normal sunlight to synthesize carbohydrates in their chlorophyll-containing tissue (photosynthesis), light stimulates the formation and release of our own chemical compounds. In light therapy, the monochromic infrared light energy has a stimulating effect on the tissues because it increases cellular energy. The light energy becomes absorbed in the tissue, hence stimulating the metabolic process.

The tissues exposed to monochromatic light increase blood flow, thus helping to carry vitamins and nutrients into the area where they are most needed with no damage to the surrounding tissues. As a result of increased blood flow, toxins and waste bi-products are taken away from the tissue. Light therapy is also know as “photo therapy”. For instance, visible red light has a positive effect at a cellular level on living tissue. This type of light therapy is beneficial in treating conditions close to the surface. Skin-layers because of their high blood and water content absorb red light readily. Light and all electromagnetic energy travels as bundles of energy called photons. The center (nucleus) of the atom contains neurons and protons. The nucleus is surrounded by electrons moving in specific orbits. Energy, in the form of photons, is released when the electrons change orbits. It is these bundles of energy that trigger biological changes within the body. We are constantly bombarded by photons from ordinary light sources including sunlight. Monochromatic light created by the LED's of the present invention has the ability to control and concentrate these photons.

To accomplish these benefits, the following example is given. This example is merely illustrative of the principles of the present invention and is not meant to limit the invention to embodiments given in this example.

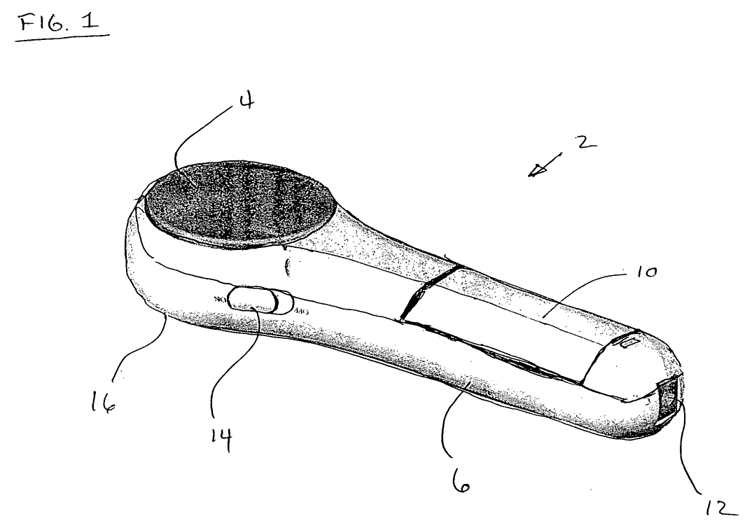

In FIG. 1, a top perspective view of a light wand 2 is shown. Light wand 2 includes a lens cover 4, which is used to protect the LEDs disposed underneath the cover. The lens cover can be clear or colored transparently, such as a blue transparent. The light wand further includes a handle 6 for gripping the wand. The wand can be any shape or design and does not necessarily require a handle to function within the scope of the present invention.

The wand 2 further includes a battery cover 10 for protecting the power source disposed beneath cover 10. Preferably the power source is four AA batteries, however an AC adapter can be used and plugged into receptacle 12. Power switch 14 turns the light wand on and off. The power output for the light wand is preferably a steady power output of about 20 milliwatts to about 50 milliwatts.

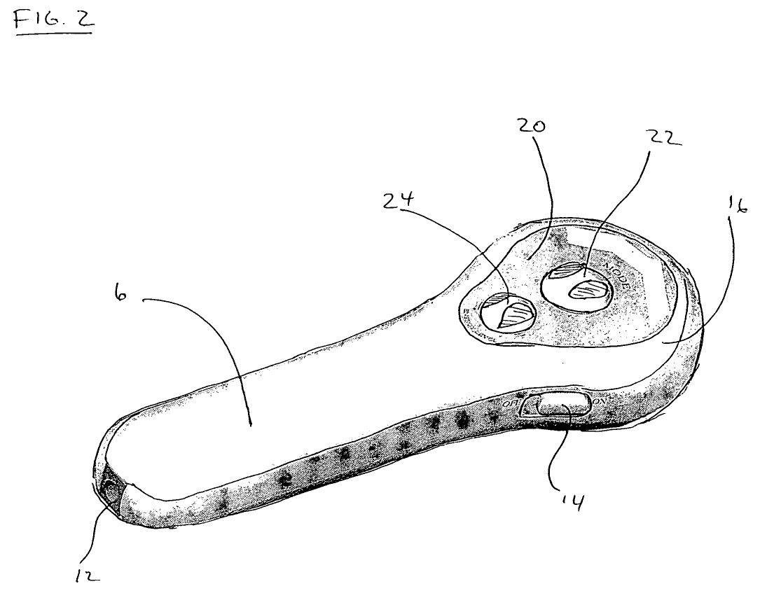

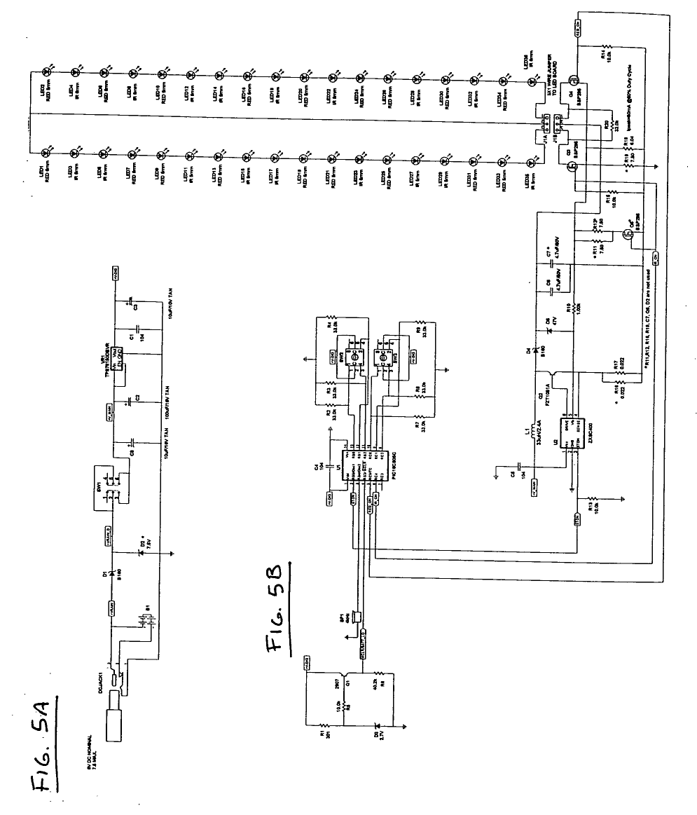

Head 16 holds the circuitry for a control unit 22 as shown in FIG. 2 and FIGS. 5A and 5B. Control unit 22 includes control switches 22 and 24. In FIGS. 5A and 5B theses switches are denoted by SW2, SW3. These control switches are for controlling the light delivery mode (pulse or concentrated stream of light or both), and the LEDs intensity level. The symbol SW1 denotes power switch 14. A microprocessor PIC18C505C further provides control of the light mode and intensity of the LEDs. In FIG. 5B, the LEDs are denoted by LED1-LED36. In this example, there are (18) red LEDs and (18) red LEDs.

It is preferred that the number of LEDs are even to provide a uniform distribution over the treatment site. However, it is within the scope of this invention to provide LEDs having a total amount that is an odd number. The distribution of the light energy will depend on the array of LEDs in the wand.

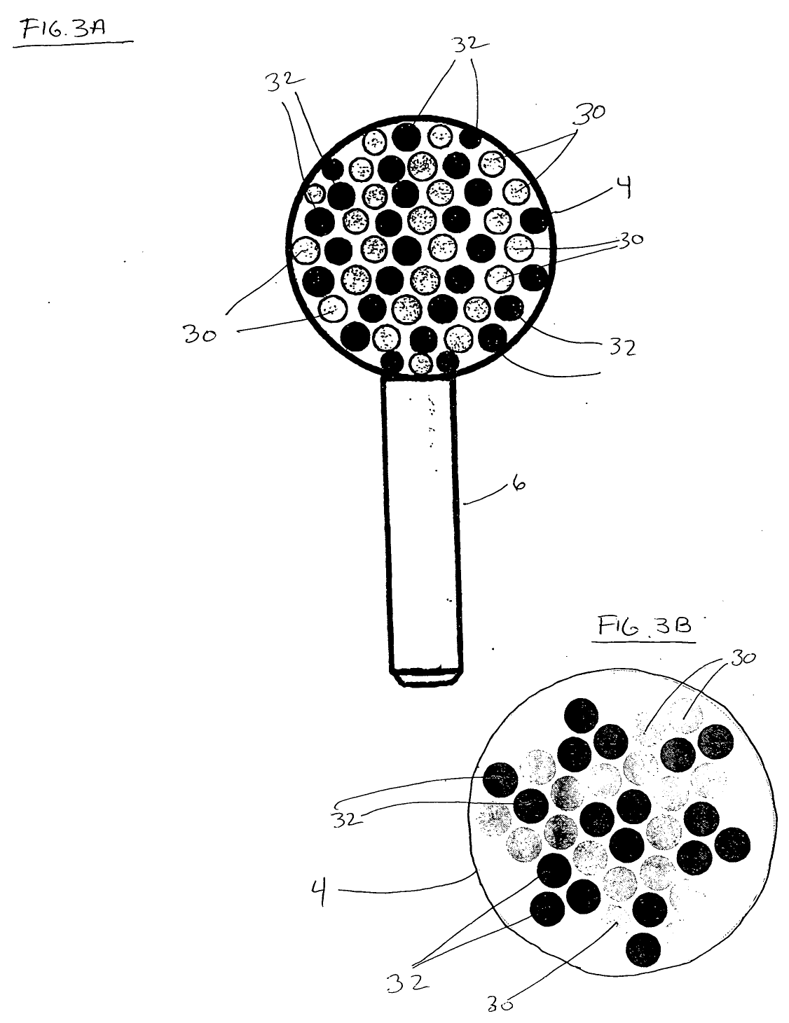

For example, as shown in FIG. 3A and FIG. 3B, two different arrays of LEDs are shown. It is preferred that the LED array is circular to allow uniform distribution of the light energy to the treatment site. Shown in these figures are red LED 30 and infrared LED 32. FIG. 3A illustrates a total number of LEDs of odd numbers (27 red, 25 infrared). FIG. 3B illustrates a total number of LEDs of even numbers (18 red, 18 infrared). Again the even numbers are preferred to distribute uniform amounts of light energy., Both arrays are in a circular pattern to further the uniform emitting of the light energy.

The LEDs are preferably red and/or infra red. The following illustration gives examples of the specifications of such LEDs. This illustration is not meant to limit the scope of the invention to this embodiment, but is merely given as an example of what LEDs can be used with the present invention for the advantages previously discussed.

TABLE 1
Examples of LEDs

| Infrared LED | Red LED |
|---|---|
| Device: Kingbright W53SF7C | Device: Kingbright W53SRC/DU |
| Wavelength: 850 nm | Wavelength: 660 nm |
| Light source distance to skin: | Light source distance to skin: 1.25 cm |
| 1.25 cm | |
| Light intensity pattern: | Light intensity pattern: |
| 1.92 Joules/cm²/minute | 1.01 Joules/cm²/minute |
| Light output energy density = | Light output energy density = |
| 1.92 Joule/cm² | 1.01 Joule/cm² |

A light wand having 18 Infrared and 18 Red LED typically provides an average light output energy density per minute of 1.46 Joules/cm². The total effective area of light out put is typically about 16 centimeters squared. Again, the round shape of the LED array assists in transmitting a uniform dosage of light energy to the treatment site without the need for a user to move the light wand back and forth to apply a uniform dosage of the LED light energy.

The light wand gives a user the ability to choose from at least 7 frequencies and at least 8 energy densities. The light frequencies are restricted to the range where body tissue can have full absorption, which is between about 600 nm to about 980 nm. Preferably the frequencies are set at about 660 nm for red LED and about 850 nm for infrared LED. The control unit provides flexibility in LED light delivery for the healing of tissue. Again, cell tissue responds best to certain frequencies that appear to be with the infrared spectrum, such as 660 nanometers (nm), 660 nm, 880 nm, 940 nm and 950 nm. The water and blood content within the body tissue restrict full absorption of light frequencies outside the range of 600-980 nm.

One frequency of light, a single wavelength that is preferably 660 nm is the most resonant frequency to the human tissue. An application of a 660 nm light beam for several minutes to a wound approximately the size of a half of dollar every two hours can (within a day or two) stimulate the generation of new skin without scabbing or forming scar tissue.

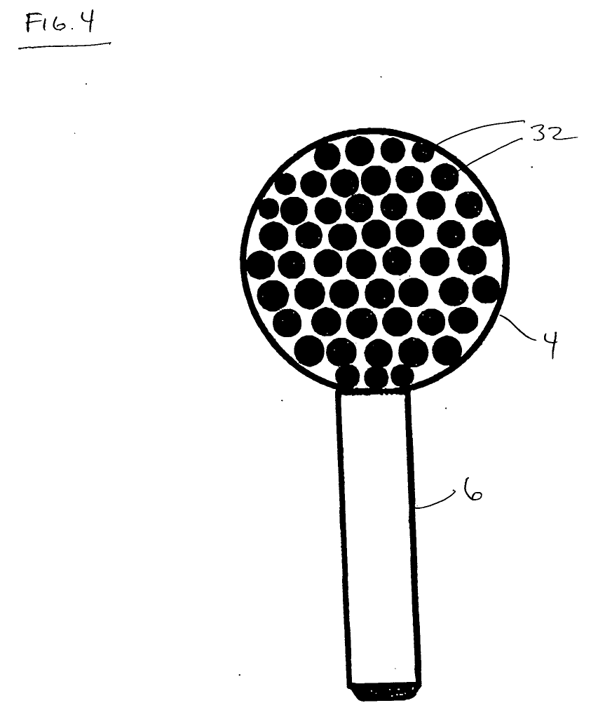

FIG. 4 illustrates a light wand having red LEDs only. In this illustration red LEDs 32 are mounted about head 4 in a circular array. The LEDs can have a total number that is odd or even. Again, an even number of LEDs is preferred to assist in the uniform distribution of light energy.

The energy intensity of the light wand has at least (8) settings. Typical average energy light output per minute is estimated at about 1.3 Joules/cm squared/minute. The following table illustrates and exemplary energy level settings of the present invention. This example is given for illustration purposes only and is not meant to limit the invention to this particular illustration.

TABLE 2
Treatment Time Per Energy Settings

| Energy Level Settings | Treatment Time |
|---|---|
| 1 Joule/cm² | 41 seconds |
| 2 Joule/cm² | 1 minute 22 seconds |
| 3 Joule/cm² | 2 minutes 3 seconds |
| 4 Joule/cm² | 2 minutes 44 seconds |
| 5 Joule/cm² | 3 minute 25 seconds |
| 15 Joule/cm² | 10 minutes 25 seconds |
| 20 Joule/cm² | 13 minutes 40 seconds |
| 30 Joule/cm² | 20 minutes 30 seconds |

The above example demonstrates that the energy level settings may have a temporal relationship with the treatment time. For example, in this illustration there is an underlying constant level of light energy and the intensity is determined by the time of exposure. The exposure time is controlled by the control unit circuitry previously described above and shown in detail in FIGS. 5A and 5B. An automatic shut off feature is included in the circuitry to avoid under and over exposure of the light energy.

The automatic shut off feature is not a typical shut off circuit. Through the microprocessor, the automatic shut off feature consistently provides control over the LEDs to emit the desired intensity and frequency of light energy. This feature prevents over or under exposure of the treatment site.

Light therapy from the light wand is emitted either in a pulsed or a constant light stream or beam or beams. The automatic shut off is provided in the control unit (microprocessor circuitry) to avoid over or under exposure of the treatment site. This automatic shut off turns the device off when the desired intensity is reached. This device is controlled by a microprocessor shown in FIG. 5B that prevents over and under exposure to the treatment site. The automatic shut off feature through the microprocessor adjusts the timing of exposure as the internal or external power source weakens or fluctuates. For example, with an internal battery source, as the battery weakens, the microprocessor adjusts the time of exposure so that the desired intensity of the LEDs is consistently achieved. If the power source is too weak to deliver the desired intensity, the device warns the user of this condition, and will prevent delivery of an undesirable dose. Similarly, if the power source is too high, the control unit will decrease the time of exposure so that the desired frequency and intensity is achieved without over exposure to the treatment site. The present invention also has the ability to control time of light energy delivery and intensity of light energy delivery.

The intensity of the light energy may be controlled by the control unit, in particular through SW3 in FIG. 5B (control switch 24) through a direct correlation in power available to the LEDs. For example, with this power supply method as the control switch 24 is increased, the power available to the LEDs is increased. This increase in power will increase the intensity of the treatment. The control unit will prevent any over or under exposure to the treatment site allowing consistent delivery of the desired Joules of light energy.

The description of operation includes turning on power switch 14. Either the AC adapter can be used or the battery pack located inside the light wand. The user then selects what mode to deliver. The mode refers to the type of light delivery, pulsed, constant stream of light or a combination of both.

After the mode is selected, the intensity may be selected. There is no limitation in the order of using the control switches in the control unit. Any sequence of steps can be used in the present invention.

The user then applies the wand over the treatment site. The wand automatically shuts off after the selected treatment intensity is delivered. The automatic shut off protects the treatment site from being over exposed. An electric timer integrated in the microprocessor of the control unit prevents the treatment site from being under exposed. This feature provides consistent treatment exposure to the treatment site without the user induced error or speculation on how long to treat the treatment site.

It should be understood that the above description is only representative of illustrative examples of embodiments. For the reader's convenience, the above description has focused on a representative sample of all possible embodiments, a sample that teaches the principles of the invention. Other embodiments may result from a different combination of portions of different embodiments. The description has not attempted to exhaustively enumerate all possible variations.

Furthermore, since numerous modifications and variations will readily occur to those skilled in the art, it is not desired that the present invention be limited to the exact construction and operation illustrated. Accordingly, all suitable modifications and equivalents that may be resorted to are intended to fall within the scope of the claims.

1. A light wand for healing tissue, comprising:
a plurality of monochromatic light emitting diodes,
a power source; and
a control unit coupled to the light emitting diodes and the power source for selectively controlling the light emitting diodes to emit light energy.

2. The device of claim 1, wherein the light emitting diodes are red diodes, infra-red diodes, or any combination thereof.

3. The device of claim 2, wherein the red diodes have at wavelength of about 660 nanometers.

4. The device of claim 2, wherein the infrared diodes have a wavelength of about 850 nanometers.

5. The device of claim 1, further including a covering lens disposed over the diodes to protect the diodes.

6. The device of claim 1, wherein the control unit further includes an automatic shut off feature to prevent over and under exposure of a treatment site of user selected light energy.

7. The device of claim 1, wherein the power source is either from a direct current or an alternating current.

8. The device of claim 1, wherein the control unit includes a circuit for the diodes to produce a pulsed stream of light energy, a constant stream of light energy, or any combination thereof to produce a therapeutic response.

9. The device of claim 8, wherein the light energy from the plurality of monochromatic light emitting diodes is from a plurality of monochromatic red diodes and a plurality of monochromatic infrared diodes that alternatively powered on and off to allow only one type of diode to be turned on at a time.

10. The device of claim 8, wherein the monochromatic light has a frequency between about 600 nanometers to about 980 nanometers.

11. A light wand for healing tissue, comprising:
a plurality of monochromatic light emitting consisting of red diodes, infrared diodes or any combination thereof,
a power source; and
a control unit coupled to the light emitting diodes and the power source for selectively controlling the light emitting diodes, the control unit having an intensity control for controlling the intensity of light energy emitted from the diodes and a mode control for providing a pulsed stream of light energy or a constant stream of light energy.

12. The device of claim 11, wherein the intensity control further includes a temporal control circuit for controlling light energy intensity from the monochromatic diodes by controlling the amount of time the diodes are turned on.

13. The device of claim 11, wherein the intensity control further includes a resistor circuit for decreasing or increasing power to the monochromatic diodes.

14. The device of claim 11 wherein the controller further includes an automatic shut off that terminates energy delivery after a predetermined time period.

15. The device of claim 11, wherein the average light output energy density of the light emitting diodes is up to about 2.0 Joule/cm².

16. The device of claim 11, wherein the average light output energy density of the red diodes is about 1.01 Joule/cm².

17. The device of claim 11, wherein the average light output energy density of the infrared diodes is about 1.92 Joule/cm².

18. The device of claim 11, wherein the total number of infrared diodes is an odd number and arranged in a circular array.

19. The device of claim 11, wherein the total number of infrared diodes is an even number and arranged in a circular array.

20. The device of claim 19, wherein the total number of infra red diodes is eighteen and the total number of red diodes is eighteen.

21. A light wand for healing tissue, comprising:
a head containing a plurality of monochromatic infrared light emitting diodes, and a lens cover disposed over the diodes;
a base connected to the head for containing a power source; and
a control unit disposed in the head and coupled to the light emitting diodes and the power source for selectively controlling the light emitting diodes, the control unit having an intensity control for controlling the intensity of light energy emitted from the diodes and a mode control for providing a pulsed stream of light energy or a constant stream of light energy.

22. A light wand for healing tissue, comprising:
a plurality of monochromatic light emitting diodes,
a power source from either direct or alternating current; and
a control unit coupled to the light emitting diodes and the power source for selectively controlling the light emitting diodes to emit light energy,
wherein the control unit controls time of exposure to the light energy for preventing over and under exposure of light energy to the treatment site based on a desired dosage selected by a user.

23. The device of claim 22, wherein the control unit further includes a microprocessor that controls energy delivery of the light energy from the light emitting diodes.

24. The device of claim 23, wherein the energy delivery is either a pulsed stream or a constant stream of light energy or any combination thereof.

25. The device of claim 22, wherein the control unit further includes a microprocessor that controls adjustments in power due to power fluctuations in the power source to prevent over and under exposure of desired dosage of light energy from the light emitting diodes.

26. The device of claim 22, wherein the control unit further includes a control switch to adjust the intensity of energy from the light emitting diodes.

27. The device of claim 22, wherein the control unit further includes a control switch to adjust the frequency of the light energy from the light emitting diodes.

28. The device of claim 22, wherein the control unit further includes a microprocessor to control the intensity of energy from the light emitting diodes.

29. The device of claim 22, wherein the control unit further includes a microprocessor to control the frequency of energy from the light emitting diodes.
